(19) United States
(12) Patent Application Publication
(10) Pub. No.: US 2010/0300882 A1
(43) Pub. Date: Dec. 2, 2010

(54) DEVICES AND METHODS FOR IN-LINE SAMPLE PREPARATION OF MATERIALS

(75) Inventors: Anping Zhang, Rexford, NY (US); Anthony John Murray, Lebanon, NJ (US); Rui Chen, Clifton Park, NY (US); Wei Yuan, Acton, MA (US)

Correspondence Address: GENERAL ELECTRIC COMPANY;GLOBAL RESEARCH, ONE RESEARCH CIRCLE, BLDG. K1-3A59, NISKAYUNA, NY, 12309, US

(73) Assignee: GENERAL ELECTRIC COMPANY, SCHENECTADY, NY (US)

(21) Appl. No.: 12/471,519

(22) Filed: May 26, 2009

(51) Int. Cl. C25B 7/00; B01D 61/42; C07K 1/26

(52) U.S. Cl. 204/543; 204/627

(57) ABSTRACT

A microfluidic device for in-line sample preparation of one or more materials. The microfludic device comprises an in-line tangential flow component. The in-line tangential flow component comprises a first channel through which the sample flows; and one or more additional channels. The first channel and the one ore more channels are separated by a membrane; and wherein a differential is present between the first channel and additional channel that is separated by the membrane.

### FIELD OF INVENTION

The invention relates generally to methods and devices for sample preparation of one or more materials. One or more of the embodiments relate generally to microfluidic devices for in-line sample preparation of one or more materials.

### BACKGROUND

Sample preparation is required for accurate and reproducible characterization of a variety of proteins or other biomolecules. In proteomic studies of complex samples, such as serum, plasma or cell extracts with a broad dynamic range of background biomolecules present, there is a need for high throughput means for sample preparation.

A variety of analytical techniques are available for protein analysis, including mass spectrometry, surface plasmon resonance molecule interaction studies, electrophoresis, nanowire sensing, and the like. It is often critical that interfering background molecules be removed from the sample but that the analyte of interest is present at a detectable concentration. Sample preparation methods are needed to permit the purification and concentration of small volume samples with minimal sample loss.

Protein analyses are increasingly performed at miniaturized scale. Consequently sample preparation steps are also miniaturized to provide fast turnaround, high throughput, small consumption of samples and valuable reagents and minimal losses. Novel sample preparation techniques are needed to meet these requirements for biomarker discovery and validation, drug discovery and proteomics research.

Current sample preparation techniques are not suitable for in-line protein analyses of small sample volumes with high throughput. For example, conventional dialysis membranes have been employed for protein/peptide desalting. Use of dialysis membranes is time-consuming and requires a large sample volume. Time-consuming sample preparation steps may increase the risk of loss of proteins that are sensitive to degradation. Another commonly used approach is to centrifuge the samples on an ultra-filtration membrane followed by dilution of the retentate. This can be repeated as a means to remove small molecule below the cut-off molecular weight. This approach could result in significant protein loss and also is time-consuming. To address these issues, several more products have become commercially available. These products can be divided into two categories, desalting pipette tips and desalting columns. The desalting columns require a large volume and a large elution volume. The pipette tip can process small sample volume, but it is performed offline and requires elution of bound proteins. In most applications, the desalting requires multiple manual-handling steps.

In-line microdialysis devices are known, but these units are relatively large, which results in large dead volume and high eluate sample volumes. Another technique that is employed to effectively desalt, purify, and concentrate proteins/peptides, is the solid phase extraction technique, which uses hydrophilic, affinity, ion exchange and hydrophobic interactions. However, this technique suffers from relatively low capacity and large elution volume, requiring time for diffusion/adsorption or resulting in low protein/peptide recovery. It is also difficult to remove contaminant particles or precipitates because the sample is loaded and eluted from the same side. In-line size exclusion chromatography (SEC) is employed to desalt and buffer exchange a protein complex according to the molecule weight. However, the separation capacity of SEC is typically poor, limiting salt removal, especially when the salt concentration is high.

Microfluidic devices have emerged to address these challenges. Microfluidic devices enable continuous flow operations with precise control and manipulation of small sample volumes. For example, microfluidic devices may be designed to perform parallel processes without manual intervention by providing a capability to perform hundreds of operations (e.g. mixing, separating, etc.).

While the applications of such microfluidic devices may be virtually boundless, the integration of some microscale components into microfluidic systems has been technically difficult, thereby limiting the range of functions that may be accomplished by a single device or combination of devices. In addition, when dealing with small volume samples, one of the major problems is a loss of sample due to the transfer of samples to and from the microfluidic devices. When sample is present in such a small volume, recovery of analyte(s) becomes an important consideration.

Therefore there exists a need to have a miniaturized device for sample preparation and methods for using the device in line. There also exists a need to have an in-line device that would effectively desalt, fractionate, and concentrate the biomolecules such as proteins, peptides, nucleic acids and the like without denaturing and /or destroying the sample.

### BRIEF DESCRIPTION

One aspect of the invention provides a microfluidic device for in-line sample preparation of one or more materials. The microfluidic device comprises an in-line tangential flow component. The in-line tangential flow component comprises a first channel through which the sample flows; and one or more additional channels. The first channel and the one or more additional channels are separated by a membrane; and wherein a differential is present between the first channel and additional channel that is separated by the membrane.

According to another aspect of the invention, a microfluidic device for in-line sample preparation of one or more materials is provided. The microfludic device comprises an in-line tangential flow component. The in-line tangential flow component comprises a first channel through which the sample flows; and one or more additional channels. The first channel and the one or more additional channel are separated by a membrane; and wherein an ionic differential is present between the first channel and additional channel that is separated by the membrane.

According to another aspect of the invention a microfluidic device for in-line concentration of one or more materials is provided. The microfluidic device comprises an in-line tangential flow component. The in-line tangential flow component comprises a first channel through which the sample flows; and one or more additional channels. The first channel and the one or more additional channels are separated by a membrane; and wherein an electrical differential is present between the first channel and additional channel that is separated by the membrane.

According to another aspect of the invention, a method for in-line sample preparation of one or more materials is provided. The method comprises providing a microfluidic device comprising an in-line tangential flow component. The in-line tangential flow component comprises a first channel through which the sample flows; and one or more additional channels. The first channel and the one or more additional channels are separated by a membrane; and wherein a differential is present between the first channel and additional channel that is separated by the membrane. The method further comprises introducing the sample feed in the first channel and allowing the sample feed to flow in a tangential manner from the first channel to the one more additional channel through the porous membrane based on the differential.

### BRIEF DESCRIPTION OF DRAWING

FIG. 1 is a cross-sectional view of a device for in-line sample preparation of one or more materials according to one embodiment of the invention.

FIG. 2 is a cross-sectional view of a device for in-line sample preparation of one or more materials according to one embodiment of the invention.

FIG. 3 is a cross-sectional view of a device for in-line sample preparation of one or more materials according to one embodiment of the invention.

FIG. 4 is a plot of the fluorescence signal versus time for in-line desalting of one or more materials according to one embodiment of the invention.

FIG. 5 is a plot of the pore size distribution of the membrane according to one embodiment of the invention.

FIG. 6 is a cross-sectional view of a device for in-line sample preparation of one or more materials according to one embodiment of the invention.

These and other features, aspects and advantages of the present invention will become better understood when the following detailed description is read with reference to the accompanying figures.

### DETAILED DESCRIPTION

To more clearly and concisely describe and point out the subject matter of the claimed invention, the following definitions are provided for specific terms, which are used in the following description and the appended claims. Throughout the specification, exemplification of specific terms should be considered as non-limiting examples. The precise use, choice of reagents, choice of variables such as flow rates, concentration, sample volume, and the like may depend in large part on the particular application for which it is intended. It is to be understood that one of skill in the art will be able to identify suitable variables based on the present disclosure. It will be within the ability of those skilled in the art, however, given the benefit of this disclosure, to select and optimize suitable conditions for using the methods in accordance with the principles of the present invention, suitable for these and other types of applications.

In the following specification, and the claims that follow, reference will be made to a number of terms that have the following meanings. The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Similarly, “free” may be used in combination with a term, and may include an insubstantial number, or trace amounts while still being considered free of the modified term.

As used herein, the term “antibody” refers to an immunoglobulin that specifically binds to and is thereby defined as complementary with a particular spatial and polar organization of another molecule. The antibody may be monoclonal or polyclonal and may be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgGI, IgG2a, IgG2b and IgG3, IgM. Functional antibody fragments may include portions of an antibody capable of retaining binding at similar affinity to full-length antibody (for example, Fab, Fv and F(ab′)₂, or Fab′). In addition, aggregates, polymers, and conjugates of immunoglobulins or their fragments may be used where appropriate so long as binding affinity for a particular molecule is substantially maintained.

As used herein, the term “peptide” refers to a sequence of amino acids connected to each other by peptide bonds between the alpha amino and carboxyl groups of adjacent amino acids. The amino acids may be the standard amino acids or some other non standard amino acids. Some of the standard nonpolar (hydrophobic) amino acids include alanine (Ala), leucine (Leu), isoleucine (Ile), valine (Val), proline (Pro), phenylalanine (Phe), tryptophan (Trp) and methionine (Met). The polar neutral amino acids include glycine (Gly), serine (Ser), threonine (Thr), cysteine (Cys), tyrosine (Tyr), asparagine (Asn) and glutamine (Gln). The positively charged (basic) amino acids include arginine (Arg), lysine (Lys) and histidine (His). The negatively charged (acidic) amino acids include aspartic acid (Asp) and glutamic acid (Glu). The non standard amino acids may be formed in body, for example by posttranslational modification, some examples of such amino acids being selenocysteine and pyrolysine. The peptides may be of a variety of lengths, either in their neutral (uncharged) form or in forms such as their salts. The peptides may be either free of modifications such as glycosylations, side chain oxidation or phosphorylation or comprising such modifications. Substitutes for an amino acid within the sequence may also be selected from other members of the class to which the amino acid belongs. A suitable peptide may also include peptides modified by additional substituents attached to the amino side chains, such as glycosyl units, lipids or inorganic ions such as phosphates as well as chemical modifications of the chains. Thus, the term “peptide” or its equivalent may be intended to include the appropriate amino acid sequence referenced, subject to the foregoing modifications, which do not destroy its functionality.

Proteins (also known as polypeptides) are organic molecules comprised of amino acids joined by peptide bonds between the carboxyl and amino groups of adjacent amino acid residues. Although proteins are linear polymers, they fold into three-dimensional structures important to their function.

As used herein, the term “enzyme” refers to a protein molecule that can catalyze a chemical reaction of a substrate. In some embodiments, a suitable enzyme catalyzes a chemical reaction of the substrate to form a reaction product that can bind to a receptor (e.g., phenolic groups) present in the sample or a solid support to which the sample is bound. A receptor may be exogeneous (that is, a receptor extrinsically adhered to the sample or the solid-support) or endogeneous (receptors present intrinsically in the sample or the solid-support). Examples of suitable enzymes include peroxidases, oxidases, phosphatases, esterases, and glycosidases. Specific examples of suitable enzymes include horseradish peroxidase, alkaline phosphatase, β-D-galactosidase, lipase, and glucose oxidase. One or more embodiments are directed to a microfluidic device for sample preparation of one or more materials. The microfluidic device has an in-line tangential flow component; wherein the in-line tangential flow component comprises a first channel through which a sample flows; and one or more additional channels.

In some embodiments, the in-line tangential flow component comprises among others a membrane. In one embodiment, the membrane separates the first and the one or more additional channels of the in-line tangential flow component. Different materials may be used as the substrate for the membrane. In one non-limiting embodiment, the substrate may be an insulator or a semiconductor, such as silicon or silicon dioxide or any combination of these materials.

In one embodiment, the membrane may be made of an inorganic material, such as silicon, or silicon nitride. The silicon nitride membrane may be amorphous in nature. In one embodiment, the membrane may be made of low-stress silicon nitride. The residual-stress of silicon nitride may be controlled by the deposition process. In one embodiment, the silicon nitride may be deposited by methods such as low-pressure chemical vapor deposition (LPCVD), plasma-enhanced chemical vapor deposition (PECVD) and the like. In one embodiment, the film stress may be less than about 250 Mpa. In another embodiment, the film stress may be less than about 50 MPa. In some embodiments where the membrane is made of silicon, the membrane may be formed of single crystal silicon, poly-crystalline silicon or amorphous silicon. The membrane formed of single crystal silicon may exhibit enhanced mechanical strength and robustness. Trans membrane pressure acceptable in case of single-crystal silicon membranes may be about 5.6 atmospheres for a 100 nanometer thick single crystal silicon membrane having a membrane size of 100 microns by 100 microns. In one example embodiment, the trans-membrane pressure in silicon nitride membranes may be about 4.3 atmospheres for a 100 nanometers thick silicon nitride membrane having a membrane size of 100 microns by 100 microns. As used herein, the term “trans-membrane pressure” refers to maximum pressure differential across the membrane before the membrane ruptures due to pressure experienced by the membrane.

In some embodiments the membrane may comprise a plurality of membranes. In one embodiment, the size of the plurality of membranes may be tuned for membrane robustness. In one embodiment, the plurality of membranes may enhance the membrane strength and robustness. In one embodiment, the membrane may be accessed from the support side by standard photolithographic patterning, followed by plasma etch or wet chemical etch of the support. In another embodiment, the membrane may be accessed from an anodized substrate. As used herein, the term “anodized substrate” refers to a substrate that comprises pores formed by anodization of the substrate. In one embodiment, the plurality of membranes may be have different shapes such as for example the plurality of membrane may be circular, rectangular, or square. In an example embodiment, the plurality of membranes may have a pore size in a range from about 1 micrometer to about 1 centimeter, or from about 50 micrometers to about 500 micrometers. In certain embodiments, the membrane comprising a plurality of membranes may have a diameter of up to about 12 inches.

Proteins and other molecules with different molecular weight may be differentiated using different pore sizes. In one embodiment, the funtionalization of the membrane may help to modulate the properties of the membrane. In a non-limiting embodiment, the functionalization of the pore surfaces of the membrane may be used to change the effective pore size; to modify the charge of the pore to be neutral, positive or negative; to minimize the non-specific adsorption of the surface; or to change the wetting properties of the membrane.

In one embodiment, the effective pore size of the membrane may be reduced by functionalization of the membrane with molecules of sufficient size to modify the pore size. Non-limiting examples of such molecules are polymers or oligomers of polyethylene glycol or proteins such as bovine serum albumin.

Pore charge may be modified by functionalization with polymers (for example acrylamide, polyethylene oxide, and the like) such as those that have been used to modify surface charge to minimize electroosmotic flow in electrophoresis. The pore charge may be modified to exhibit positive charge by modification with amine functional groups for example. Negatively charged pores may result from silicon dioxide coated pores, although such pores may be additionally functionalized with compounds such as for example carboxylic acid. The charge modification of the pores may allow for additional selectivity of nanoporous membranes, although charge shielding due to sample ionic strength or pH will modulate these effects.

In some cases, there may be a need to minimize non-specific adsorption on the membrane surface and pore surfaces in order to reduce losses of the molecules of interest. Non-limiting examples of molecules employed to reduce non-specific adsorption of proteins are polymers such as polyethylene glycol.

Functionalization of the membrane with molecules that reduce the surface tension of the membrane surfaces may assist in the wetability characteristics of the membrane. Functionalization of the surface with hydrophilic polymers or oligomers such as polyethylene glycol, acylamide etc. may improve wetability or hydrophilicity of membrane surfaces.

In some embodiments, the membrane comprises a plurality of pores. In some embodiments, the size of the pores may be in a range from about 5 nanometers to about 50 micrometers. For sample preparation of proteins, the pores are referred as “nanopores”. Large pores in the membrane may be used to differentiate cells, bacteria, or other large biomolecules or aggregates. In some embodiments, the size of the pores may be in a range from about 10 nanometers to about 50 nanometers for sample preparation of proteins. In one embodiment, the thickness of the membrane may be in a range from about 5 nanometers to about 1000 micrometers. In another embodiment, the thickness of the membrane may be in a range from about 10 nanometers to about 50 nanometers, from about 50 nanometers to about 100 nanometers, from about 100 nanometers to about 500 nanometers. Thickness uniformity is better than 5%. A thin membrane reduces transport resistance across the membrane and enables high flux rate. A combination of high flux rate with narrow pore size distribution enables such a membrane for in-line protein fractionation, protein purification, protein desalting, protein concentration, and the like. In one example, the membrane may be a silicon membrane having a thickness of about 40 nanometers. In another example, the membrane may be a silicon nitride membrane having a thickness of about 50 nanometers. In some embodiments, the membrane has a porosity in a range from about 1 percent to about 90 percent. In one example, the single-crystal silicon membrane has a porosity of 10%.

In one embodiment, the membrane has a size in a range from about 1 micron to about 1 centimeter in diameter. The membrane may be made into various shapes and configurations, such as but not limited to, membranes that are square, rectangular, or elongated ovals. In some embodiments, the membrane has a size of less than about 100 micrometers. Decreasing the membrane area may increase the robustness of the membrane.

In one example embodiment, the device may be employed for sample preparation of biomolecules including, but not limited to, protein desalting. As will be appreciated, efficient protein desalting is a required preparation step for many biological samples. As used herein, the term “biological sample” refers to a sample obtained from a biological subject, including samples of biological tissue or fluid origin obtained in vivo or in vitro. Such samples can be, but are not limited to, body fluid (e.g., blood, blood plasma, serum, or urine), cell extracts, or tissue extracts. Biological samples could also include peptides, proteins, enzymes, nucleotides, nucleic acid, and the like. The desalted samples may then be used for a variety of downstream proteomics applications including but not limited to mass-spectroscopy, surface plasmon resonance (SPR), electrophoresis (on-line), process analytical technologies (PAT), enzymatic assay separation, and nanowire based protein sensing.

In one example, the tangential flow component may be coupled to down-stream detection technologies for in-line or on-chip desalting prior to the protein detection. The in-line sample preparation device may provide properties that facilitate in-situ protein analysis. For example, properties such as narrow pore distribution, fast desalting rate, high flux rate, and minimized sample loss are some of the properties that are provided by the low thickness membranes. Conventional polymer or ceramic-based membranes suffer from slow filtration rate due to high thickness (typically greater than about 100 microns), broad pore size distribution and filtration loss within the membrane. Further, it is difficult to integrate conventional membranes for in-line or on-chip applications. The tangential flow component may be fabricated to have a combination of mechanical integrity and fast desalting rate.

Protein or peptide desalting may either involve desalting one or more ions from biological fluids or sample such as for example serum. As will be appreciated, protein desalting is vital for the characterization of the function, structure, and interactions of the protein of interest. The starting material is usually a biological tissue or a microbial culture. The various steps in the desalting process may free the protein from a matrix that confines it, separate the protein and non-protein parts of the mixture, and finally separate the desired protein from all other proteins. Desalting steps exploit differences in protein size, physico-chemical properties and binding affinity. In one embodiment, at least a portion of the membrane may be functionalized to increase the affinity of the membrane for a particular type of protein, for example. Small pore size distribution of the membrane facilitates desalting without losing many of the small molecular weight proteins.

In some embodiments, the tangential flow component comprises a first channel. In one embodiment, the first channel may have at least one inlet and at least one outlet. In another embodiment, the tangential flow component comprises one ore more additional channels. In one embodiment, the one or more additional channels may have at least one inlet and at least one outlet.

In one embodiment, the first channel and the one or more additional channels of the tangential flow component may comprise a material that may be an organic, an inorganic or any combination therefrom. In some embodiments, the material may be a polymer material. Polymers may include, but are not limited to polydimethylsiloxane (PDMS). Other choices include polystyrene, poly(tetra)fluoroethylene (PTFE), polyamide, polyester, polyvinylidenedifluoride, polycarbonate, polymethylmethacrylate, polyacrylonitrile (PAN), polyvinylethylene, polyethyleneimine, poly(etherether)ketone, polyoxymethylene (POM); polyvinylphenol; polylactides; epoxy polymer such as for example SU8 photoreists, polymethacrylimide (PMI); polyalkenesulfone (PAS); polypropylene; polyethylene, polyhydroxyethylmethacrylate (HEMA), poly(ethylene terephthalate) (PETG), polyaniline, metal-organic polymers, polydimethylsiloxane (PDMS), polyacrylamide, polyimide, blends, copolymers and combinations of any of the foregoing. Non-limiting examples of the inorganic materials include silicon, silica, quartz, glass, anodic aluminum oxide, silicon nitride, and the like.

The dimensions of the first channel and the one or more additional channels may vary. However, in microfluidic embodiments the scale is small enough so as to only require minute fluid sample volumes. In some embodiments, the width and depth of the first channel and one or more additional channels of the tangential flow component may be a range from about 10 μm and about 500 μm. In some embodiment of the device, the width and depth of the first channel and additional channels may be a range from about 50 and 200 μm. In one embodiment, the length of the first channel and additional channel of the tangential flow component may be a range from about 1 to about 20 mm. In some example embodiments, the length of the first channel and additional channel of the tangential flow component may be a range from about 2 to about 8 mm. In one embodiment, the first channel and additional channel cross-section geometry may be trapezoidal, rectangular, v-shaped, semicircular, etc. The geometry may be determined by the type of microfabrication or micromachining process used to generate the microchannels, as is known in the art.

In one embodiment, a pressure differential is present between the first channel and additional channel that is separated by the membrane. In another embodiment, a concentration differential is present across the membrane. In another embodiment, the differential may be an ionic differential. As used herein the term ionic differential refers to a difference in the concentration of the ions between the first channel and the additional channel that is separated by the membrane. This difference may build a concentration gradient between the first channel and the additional channel thereby facilitating the movement of the one or more molecules of interest.

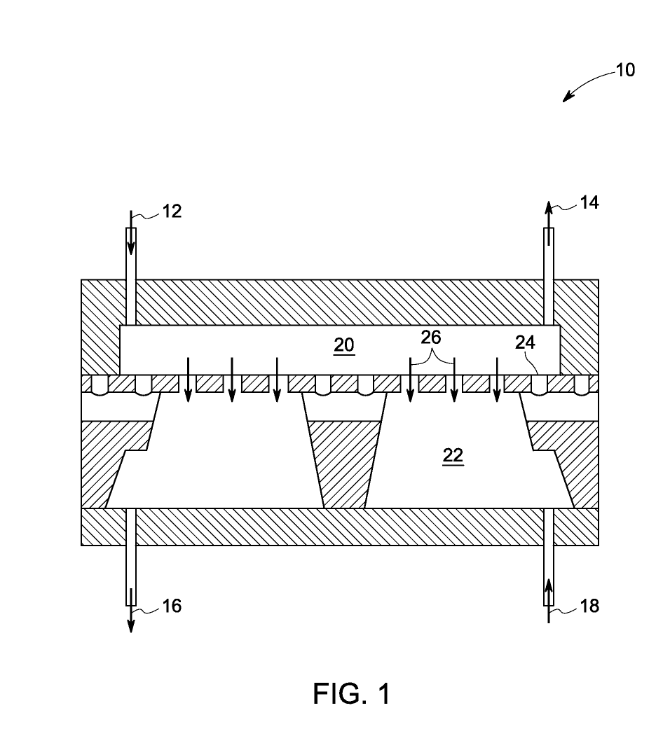

FIG. 1 illustrates the microfluidic device comprising a tangential flow component (10). The tangential flow component comprises an upper channel (20) and a lower channel (22). The upper channel and the lower channel are separated by a membrane (24). In one embodiment, the upper channel may be made of an epoxy polymer for example a SU-8 photoresist or a siloxane polymer such as polydimethylsiloxane (PDMS). In some embodiments, the lower channel is made from silicon substrate. In one embodiment, the lower channel comprises a silicon substrate capped with a polymeric material such as polydimethylsiloxane (PDMS). In one embodiment, the PDMS may contain holes that may be punched or laser drilled to connect the inlet tubing and outlet tubing. In one embodiment, the samples emerging from the outlet (14) in the upper channel and/or the outlet (16) in the lower channel may be conveyed to down-stream applications/analysis.

In one embodiment, the device of FIG. 1 may be employed for protein desalting. For desalting, a protein sample may be introduced in the upper channel (20) through the inlet on the upper channel (12) and passed through the membrane (24). A buffer with low ionic strength or water may be introduced in the lower channel via an inlet (18) in the lower channel, and passed under the membrane. In one embodiment, a counter-flow may be maintained. The ionic differential between the upper and lower channel enables the ions to flow from the upper to the lower channel (26). The outlet (14) in the upper channel may be employed to draw in the sample, and in this case, the protein out, while the outlet (16) in the lower channel may be used to draw out the buffer solution.

An example of a method of making the device is provided. The membrane may be silicon or silicon nitride membrane. It contains a plurality of nanopores that may have a pore size in a range from about 5 nanometers to about 500 nanometers, or from about 10 nanometers to about 50 nanometers. The pores may be fabricated by methods such as but not limited to, self-assembly of block copolymers, or nano-imprint. Typically, block copolymers are two different polymer chains covalently bonded together on one end and molecular connectivity may force phase separation to occur on molecular-length scales. As a result, periodically ordered structures, such as cylinders, may be formed. The cylinders may be of nanometer size. The sizes and periods of the cylinders may be governed by the chain dimensions of the block copolymers. Further, the sizes and periods of the cylinders may be of the order of about 10 nanometers to about 50 nanometers. Although, structures smaller than about 10 nanometers may also be obtainable if appropriate blocks are chosen. For example, blocks of the copolymer with a high Flory-Huggins interaction parameter and decreased block lengths may be used to obtain structures smaller than about 10 nanometers.

In some other embodiments, SU-8 photoresist may be used to fabricate the top channel. SU-8 resist has different viscosities with thicknesses of 1-300 um and can be reliably spin-coated. In one embodiment, the photoresist may be exposed to UV light through a photomask, and a developer solution is used to dissolve the unexposed regions. The top channel may be capped by a flat PDMS piece. In some embodiments, the top channel may be fabricated in PDMS with a SU-8 or silicon mold. The SU-8 mold may be made by the photolithographic method described above. The silicon mold may be fabricated by a standard photolithographic patterning, followed by a reactive ion etch (RIE) step. The surface of the silicon or SU-8 mold may be then treated with fluorinated silanes to facilitate the PDMS release. A liquid PDMS prepolymer (in a mixture of about 1:10 ratio of base polymer tocuring agent) is poured on the silicon or SU-8 mold. The PDMS is cured at about 70° C. for at least about one hour and then released from the mold with the microlfuidic channel transferred from the mold. Small holes are punched or laser drilled in the PDMS layer by methods known to one skilled in the art to produce inlets and outlets. Following this the PDMS may seal to the silicon or silicon nitride membrane surfaces reversibly by conformal contact (via van der Waals forces). In one embodiment, the PDMS may seal to the silicon or silicon nitride membrane surfaces irreversibly if both surfaces are Si-based materials and have been oxidized by plasma before contact (a process that forms a covalent O—Si—O bond).

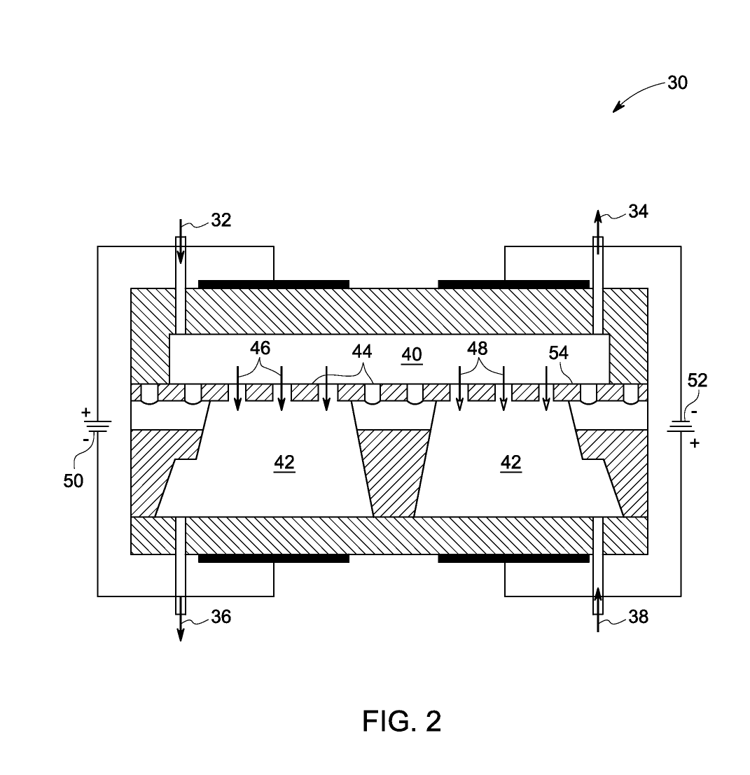

FIG. 2 is an alternate embodiment of the microfluidic device of FIG. 1 comprising the tangential flow component (30). The tangential flow component comprises an upper channel (40) and a lower channel (42). The upper channel and the lower channel may be separated by membranes (44) and (54). FIG.2 illustrates a sequential removal of positive ions (46) and negative ions (48) by the membrane. An electric field (50) may be applied across the membrane (44) that promotes the diffusion of positive ions. A reversed electrical field (52) may be applied across the membrane (54) that promotes the diffusion of negative ions. The electrical field may be employed to accelerate the diffusion process and reduce the time. FIG. 2 is a schematic representation for a 2-zone microfluidic device. The upper channel comprises an inlet (32) and an outlet (34) and the lower channel comprises an inlet (38) and outlet (36).

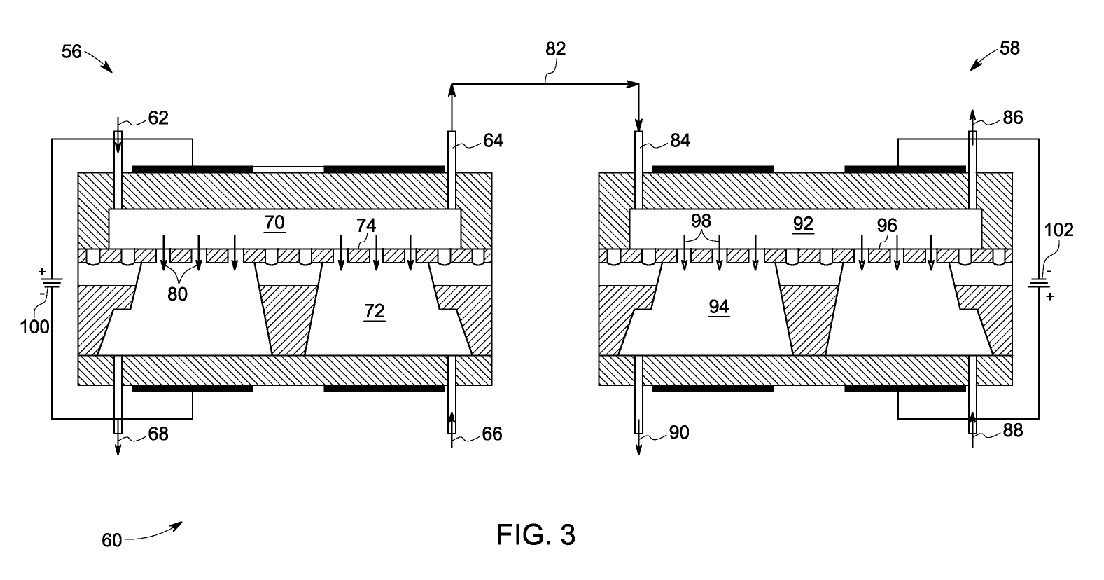

FIG. 3 is an alternate embodiment of the microfluidic device of FIG. 2 comprising the tangential flow component (60). The tangential flow component (60) comprises two tangential flow components (56) and (58) coupled to each other. The tangential flow component (56) comprises an upper channel (70) and the lower channel (72) may be separated by a membrane (74). The upper channel comprises an inlet (62) and an outlet (64) and the lower channel comprises an inlet (66) and outlet (68). An electric filed (100) may be applied across the membrane (74) that promotes the diffusion of positive ions (80) through the membrane into the lower channel. The sample after the diffusion of the positive ions (82) may be transferred into the second tangential flow component (58) via the outlet (64) in the upper channel of the tangential flow component (56) and the inlet (84) in the tangential flow component (58). Further, processing of the sample may be carried out at this point in between outlet 64 and prior to sample entering the second tangential flow component (58) via inlet 84. The tangential flow component (58) comprises an upper channel (92) and the lower channel (94) may be separated by a membrane (96). A reversed electrical field (102) may be applied across the membrane (96) that promotes the diffusion of negative ions (98).

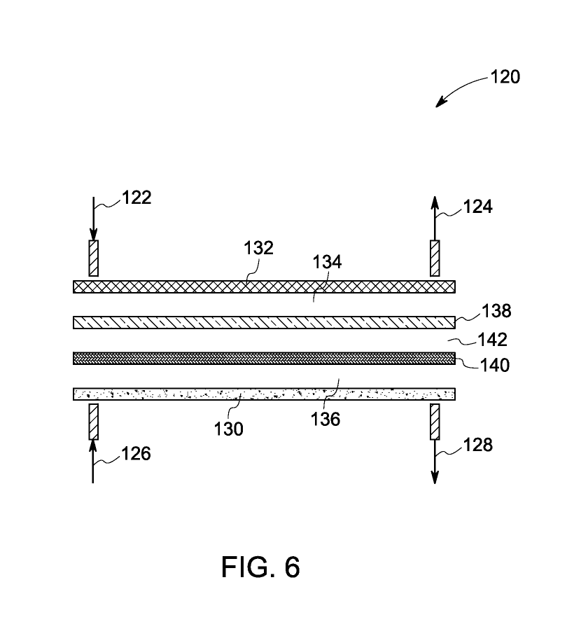

FIG. 6 is an alternate embodiment of the microfluidic device of FIG. 2 comprising the tangential flow component (120). The tangential flow component comprises a first channel (142) containing the sample, and additional channels (136 and 134). The first channel is separated from the additional channels by membranes (138 and 140). FIG. 6 illustrates the concurrent removal of positive and negative ions by the membranes. An electric field may be applied across the membranes, which promotes the diffusion of both positive and negative ions towards their respective electrodes (anode (132) and cathode (130)). The electric field may be employed to accelerate the diffusion process and thereby reduce time. In another embodiment, the tangential flow the additional channels of FIG. 6 may comprise static compartments, which may contain fluid or a pad wetted with fluid.

In one embodiment, the devices of the present invention may be employed in drug development, such as in high-throughput drug screening, medical diagnostics with body fluids (serum, plasma, etc.), biomarker discovery and validation, and the like. In some embodiments, the devices of the invention may also be useful for protein profiling in proteomics.

In one embodiment, the sides, bottom, or cover of the first channel and the one or more additional channels of the tangential flow component may be further chemically modified to achieve the required bioreactive and biocompatible properties. A wide range of detection methods either quantitative or qualitative may be interfaced to the device of the invention. In one embodiment, the microfluidic device may be interfaced with optical detection methods such as absorption in the visible or infrared range, chemoluminescence, and fluorescence (including lifetime, polarization, fluorescence correlation spectroscopy (FCS), and fluorescence-resonance energy transfer (FRET)).

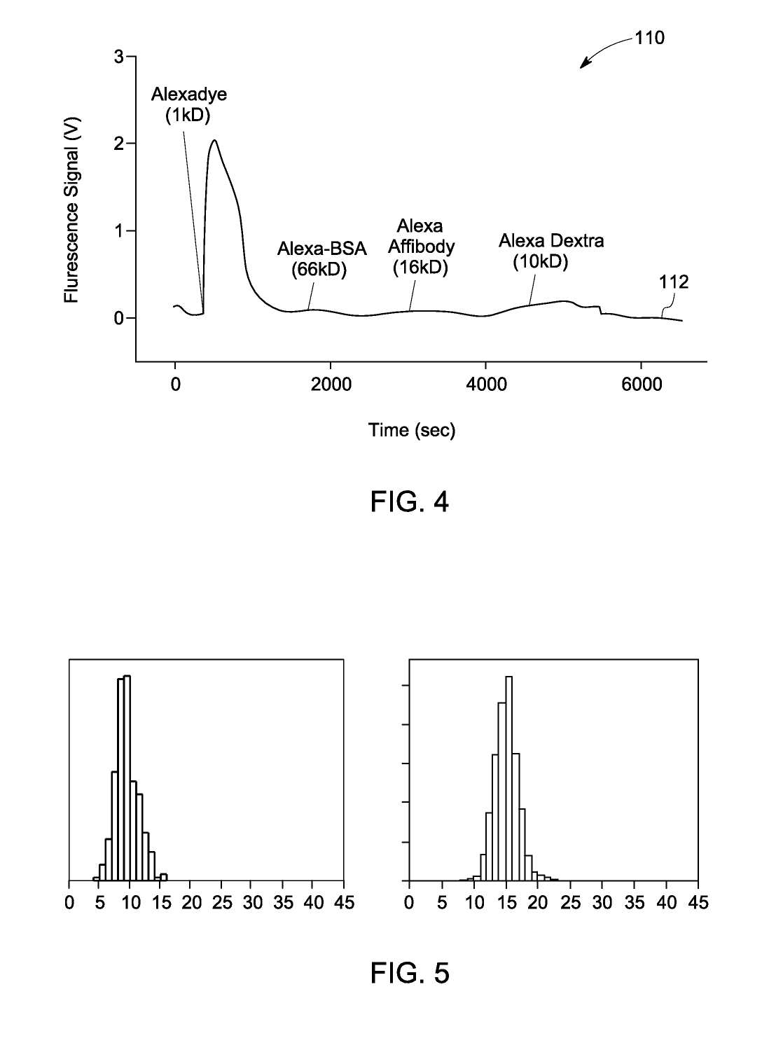

FIG. 4 illustrates the working of the microfluidic device according to one embodiment of the invention. The plot (110) of the fluorescence signal versus time is shown. The silcon membrane used in these experments was about 40 nm thick and the pore size was about 10 nm. The graph (112) is an example of the fluorescence signal (dye concentration) as function of time for Alexa dye (1 kD molecular weight), Alexa-dextran (10 kD molecular weght), Alexa-affibody (16 kD molecular weight) and Alex-BSA (66 kD molecular weight) in 5×PBS buffer. The estimated flux of Alexa dyes was found to be more than five times the flux rate of a dialysis membrane with 50 kD molecular weight cutoff. The estimated loss was 8% for Alexa-dextran (10 kD), 7% for Alexa-affibody (16 kD) and <1% for Alexa-BSA. These results indicate that the Si membrane can selectively pass the small molecules (dyes or ions) and hold the larger molecules (small or large proteins). The microfluidic devices can be used as an effective desalting device for in-line sample preparation of biomolecules.

FIG. 5 illustrates the nanopore size distribution of the membrane. It may be observed that the pore size distribution is narrow about 10-20 nanometer nanopores. A uniform pore size distribution and pore density allow a good flux rate, and the low surface to volume ratio of the membrane reduces the protein adsorptive losses.

The term “one or more materials” or “analyte” are used interchangeably. In some embodiments, the one or more materials can be determined by the type and nature of analysis required for the sample. In some embodiments, the analysis can provide information about the presence or absence of one or more materials in the sample.

In one embodiment, the one or more material may include one or more biological agents. Suitable biological agents may include pathogens, toxins, or combinations thereof. Biological agents may also include prions, microorganisms (viruses, bacteria and fungi) and some unicellular and multicellular eukaryotes (for example parasites) and their associated toxins. Pathogens are infectious agents that can cause disease or illness to their host (animal or plant). Pathogens may include one or more of bacteria, viruses, protozoa, fungi, parasites, or prions.

In one embodiment, the one or more materials, can include one or more biomolecules. In one embodiment, a biomolecule-based molecule of interest can be part of a biological agent, such as, a pathogen. In one embodiment, a biomolecule can be used for diagnostic, therapeutic, or prognostic applications, for example, in RNA or DNA assays. Suitable biomolecules can include one or more of peptides, proteins (e.g., antibodies, affibodies, or aptamers), nucleic acids (e.g., polynucleotides, DNA, RNA, or aptamers); polysaccharides (e.g., lectins or sugars), lipids, enzymes, enzyme substrates, ligands, receptors, vitamins, antigens, or haptens. The term “one or more materials” refers to both whole molecules and to regions of such molecules, such as an epitope of a protein that can specifically bind one or more antibodies or binders.

Only certain features of the invention have been illustrated and are selected embodiments from a manifold of all possible embodiments. The invention may be embodied in other specific forms without departing from the spirit or essential characteristics thereof. While only certain features of the invention have been illustrated and described herein, one skilled in the art, given the benefit of this disclosure, will be able to make modifications/changes to optimize the parameters. The foregoing embodiments are therefore to be considered in all respects as illustrative rather than limiting on the invention described herein. Where necessary, ranges have been supplied, and those ranges are inclusive of all sub-ranges there between.

1. A microfluidic device for in-line sample preparation of one or more materials comprising:
an in-line tangential flow component comprising:
a first channel through which a sample flows;
one or more additional channels;
wherein the first channel and the one or more additional channels are separated by a membrane comprising silicon, silicon nitride or combinations thereof; and
wherein a differential is present between the first channel and the additional channel that is separated by the membrane.

2. The device of claim 1, wherein the membrane has a thickness that is from about 10 to 100 nanometers and comprises a plurality of pores having a pore diameter between about 10 and 20 nanometers.

3. (canceled)

4. The device of claim 1, wherein at least a portion of the membrane is functionalized.

5. The device of claim 4, wherein the membrane is functionalized to modulate at least one of the membrane properties selected from the pore size, modify charge of the pore, adjust surface adsorption, or modulate the wetability of the membrane,

6. The device of claim 1, wherein the porous membrane has a thickness a range from about 5 nanometers to about 1000 micrometers.

7. The device of claim 1, wherein the membrane comprises a plurality of pores having a diameter a range from about 5 nanometer to about 50 micrometers.

8. The device of claim 1, wherein the membrane is between about 5 nanometers to 100 micrometers thick and has a thickness uniformity that is less than or equal to 5%.

9. The device of claim 1, wherein the membrane has a thickness from about 5 nanometers to 1000 micrometers and comprises pores having diameters in a range from about 5 nanometers to about 500 nanometers.

10. The device of claim 1, wherein the in-line tangential flow component is incorporated in a microchip.

11. The device of claim 10, wherein the differential is an electric differential.

12. A microfluidic device for in-line desalting one or more materials comprising:
an in-line tangential flow component comprising:
a first channel through which a sample flows;
one or more additional channels;
wherein the first channel and the one or more additional channels are separated by a membrane comprising silicon, silicon nitride or combinations thereof; and
wherein an ionic differential is present between the first channel and additional channel that is separated by the membrane.

13. The device of claim 12, wherein the membrane has a thickness a range from about 5 nanometers to about 1000 micrometers.

14. The device of claim 12, wherein the membrane has a pore diameter at least less than about 15 nanometers.

15. The device of claim 12, wherein the membrane comprises a plurality of membranes having a pore diameter a range from about 5 nanometer to about 50 micrometers.

16. The device of claim 12, wherein the membrane has a pore diameter a range from about 10 nanometers to about 1 micron.

17. A microfluidic device for in-line concentration one or more materials comprising:
an in-line tangential flow component comprising:
a first channel through which a sample flows;
one or more additional channels;
wherein the first channel and the one or more additional channels are separated by a membrane comprising silicon, silicon nitride or combinations thereof; and
wherein an electrical differential is present between the first channel and additional channel that is separated by the membrane.

18. A method for in-line concentration of one or more materials comprising:
providing a microfluidic device comprising:
an in-line tangential flow component comprising:
a first channel through which a sample feed flows;
one or more additional channels;
wherein the first channel and the one or more additional channels are separated by a membrane; and wherein a differential is present between the first channel and additional channel that is separated by the membrane;
introducing the sample feed in the first channel and allowing the sample feed to flow in a tangential manner from the first channel to the one or more additional channels through the porous membrane based on the differential.

19. The device of claim 1, wherein the membrane comprises a plurality of pores and wherein at least a portion of the membrane is functionalized to modify a charge of the membrane, a wetting property of the membrane, a non-specific adsorption of one or more molecules of interest or a combination thereof.

20. The device of claim 1, comprising a plurality of tangential flow components, at least two of which are microfluidic components that are operatively coupled to each other.
